(19) United States
(12) Patent Application Publication
(10) Pub. No.: US 2025/0134389 A1
(43) Pub. Date: May 1, 2025

(54) WEARABLE DEVICES FOR DETECTING EVENTS USING EXTERNAL EAR AND AMBIENT TEMPERATURE, INCLUDING EXAMPLES OF EARRINGS

(71) Applicant: University of Washington, Seattle, WA (US)

(72) Inventors: Qiuyue Xue, Seattle, WA (US); Yujia Liu, San Diego, CA (US); Vikram Subramaniam Iyer, Seattle, WA (US); Shwetak N. Patel, Seattle, WA (US)

(21) Appl. No.: 18/932,410

(22) Filed: Oct. 30, 2024

Related U.S. Application Data
(60) Provisional application No. 63/546,701, filed on Oct. 31, 2023.

(51) Int. Cl. A61B 5/01; A61B 5/00

(52) U.S. Cl. CPC A61B 5/01; A61B 5/0008; A61B 5/6803; A61B 5/7267; A61B 5/7282; A61B 5/742; A61B 2560/0214; A61B 2560/0252; A61B 2562/0271

(57) ABSTRACT

In one aspect, a wearable device includes a first temperature sensor configured to be positioned to detect an external ear temperature at an ear, and a second temperature sensor configured to dangle below the ear to detect an ambient temperature. The wearable device may include a controller coupled to the first temperature sensor and the second temperature sensor. The wearable device may also include an antenna coupled to the controller, where the antenna and the controller are configured to communicate data indicative of the external ear temperature and the ambient temperature. The communicated data can then be analyzed using a computing system to determine and/or detect an event based on the data indicative of external ear temperature and the data indicative of ambient temperature.

### CROSS-REFERENCE TO RELATED APPLICATIONS

This application claims the benefit under 35 U.S.C. § 119(e) of the earlier filing date of U.S. Provisional Application No. 63/546,701 filed Oct. 31, 2023, the entire contents of which are hereby incorporated by reference in their entirety for any purpose.

### BACKGROUND

Wearable devices with sensors have gained significant popularity in recent years and are becoming a ubiquitous part of daily life. Many users utilize one or more wearable devices, such as smartwatches, smart rings, smart jewelry, smart glasses, smart earbuds, and/or smart clothing.

Body temperature is a vital sign that can be measured or and may be relevant to a variety of outcomes. For example, elevated core body temperatures can be a symptom for many viral infections, such as influenza and/or COVID-19. Invasive thermal measuring techniques and/or devices, such as arterial catheters or e-pills can provide accurate measurements of core body temperature by entering the body of a patient or a user through arteries or the intestine. These invasive techniques and/or devices, however, may not be suitable for everyday use. Non-invasive thermal measuring techniques and/or devices, such as oral thermometers, axillary thermometers, tympanic thermistors, and/or infrared temporal thermometers, are available. The user or patient, however, often only utilizes these non-invasive thermometers periodically, for example, one or a few times per year.

Some systems may measure body temperature by incorporating temperature sensors into wearable devices, thermal cameras, smartphones, etc. For example, a user may utilize wrist-mounted temperature sensors for core body temperature sensing. Unfortunately, these temperature sensors may fail to provide accurate measurements due to, for example, noisy temperature signals from the wrist. As another example, the user may utilize infrared thermopile sensors embedded in or on headphones to monitor tympanic temperature directly and longitudinally. However, the user needs to wear these headphones to measure their body temperature, which may not be suitable in many environments. As yet another example, the user may measure their body temperature using thermal cameras on a facial video (e.g., similar to taking a “selfie”). These sensors, however, are considerably costly and may not be suitable for personalized temperature sensing applications, such as a user or patient being able to measure their body temperature at home, instead of, for example, at a clinic.

### BRIEF DESCRIPTION OF THE DRAWINGS

FIG. 1 is a schematic illustration of a system arranged in accordance with examples described herein.

FIG. 2A is a schematic illustration of blood vessels around an external ear of a user, in accordance with examples described herein.

FIG. 2B is a schematic illustration of heat distribution of an external ear, a head, and/or a neck of the user, and/or an ambient heat distribution, in accordance with examples described herein.

FIG. 3 is a flowchart of a method arranged in accordance with examples described herein.

FIG. 4 is another schematic illustration of another system arranged in accordance with examples described herein.

FIG. 5 is a schematic illustration of an earring arranged in accordance with examples described herein.

FIG. 6 is a schematic illustration of temperature results across example activities arranged in accordance with examples described herein.

### DETAILED DESCRIPTION

Wearable devices that are capable of measuring an external ear temperature (e.g., temperature of an outer ear, an auricle, an earlobe, etc.) of a user are disclosed herein. In some embodiments, the user utilizes an earring(s) to monitor changes of their core body temperature by measuring and/or monitoring the temperature of their external ear(s) (e.g., their out ear(s), their auricle(s), their earlobe(s)) because there is a strong positive correlation between the core body temperature and the temperature of their external ear(s). That is, an increase in the temperature of the core body temperature causes an increase in the temperature of the external ear(s). And, vice versa, a decrease in the temperature of the core body temperature causes a decrease in the temperature of the external ear(s). The earring(s) can also measure the ambient temperature. In some embodiments, temperature data of the auricle of the ear can be compared to the ambient temperature to determine whether the temperature changes of the auricle of the ear are driven by changes of the core body temperature or by changes of the ambient.

Examples of wearable devices described herein may advantageously be provided in the form factor of an earring (e.g., to be worn on an external ear, such as on outer ear, on an auricle of the ear, and/or an earlobe of the ear). In the United States, the majority (e.g., 70-80%) of women have pierced earlobes, which is approximately twice the current adoption rate of smartwatches. There is also a growing trend of men wearing earrings, indicating that wearing earrings may be a widespread fashion choice. In some embodiments, embedding electronic components in or on earrings may be advantageous for continuous or interval monitoring of the core body temperature because, for example, unlike headphones or earbuds, earrings are typically worn continuously for most of the day.

In some embodiments, embedding electronic components in or on earrings to measure the core body temperature may be advantageous because the temperature of the external ear is tightly coupled to a user's core body temperature. This may not be the case with other wearable devices. For example, using smartwatches to measure the core body temperature may sometimes provide false core body temperature data when the smartwatch shifts against the skin of the user, or when the user loosely wears their smartwatch. As another example, the temperature of a user's extremities (e.g., hands, wrists, fingers, toes) may not be tightly coupled the user's core body temperature, since the user's extremities are relatively far away from the core body. As yet another example, the temperature of the user's hand or finger can change when the user touches objects with a different temperature.

Generally, when a user experiences physiological, psychological, and/or emotional events, the temperature of their external ear and/or their core body temperature may change. Generally, or traditionally, users (e.g., patients) may measure their core body temperature when they are experiencing a physiological event, such as when they are feeling unwell. In such a case, the user may choose to measure their core body temperature using a thermometer orally, axillary, in-ear, by skin, etc. Taking the temperature using a traditional thermometer when the user is unwell quite rarely (e.g., one or a few times a year) may be acceptable. If the user experiences physiological events on a regular basis, however, using a thermometer to measure their core body temperature may be inconvenient, impractical, uneasy, etc. For example, some women may want to measure and/or monitor their core body temperature when they are experiencing vasomotor symptoms (VMS) of menopause and/or monitoring for ovulation for fertility. These women may want to continuously measure and/or monitor their core body temperature passively and/or somewhat discreetly. Using the earring(s) described herein may give these women a way to monitor changes of their core body temperature with ease by measuring the temperature of the external ear(s) (e.g., their out ear(s), their auricle(s), their earlobe(s)). In some embodiments, the earrings described herein may also contain an esthetic fashion appeal, which may encourage a person to wear and/or utilize the described earring(s) on a regular basis. The user can then choose (or not choose) to discuss the temperature data with their personal doctor, for example, in the privacy of the doctor's office.

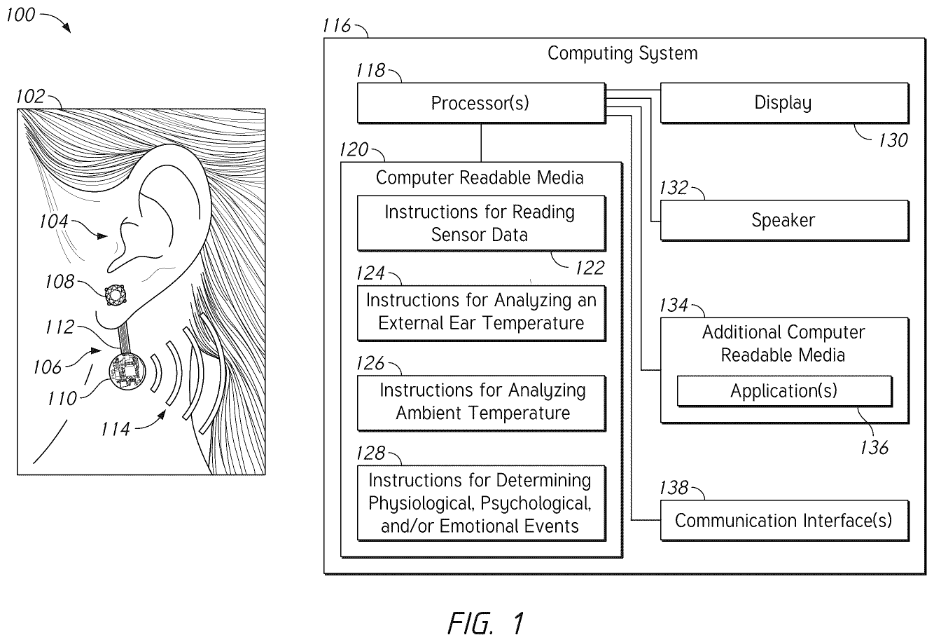

As discussed herein, the temperature of the external ear and/or the core body temperature may also change when the user experiences a psychological and/or an emotional event. For example, when the user experiences stress, is suddenly shy, or feels another psychological and/or emotional event, the user's face and/or external ears may turn “turn red” because involuntarily blood may have an increased flow (or surge) to the head, face, and/or external ears. The increased blood flow to the head, face, and/or external ears of the user may cause a change in the temperature of the head, the temperature of the face, the temperature of the external ears, corporeal temperature, and/or core body temperature, regardless of the ambient temperature. Traditionally, when a user feels these psychological and/or emotional events, they do not measure their temperature using a thermometer. Often, the user may even be unaware that their corporeal temperature and/or the temperature of their external ear(s) may have changed due to these events. The system 100 of FIG. 1, however, can measure and/or monitor the described physiological, psychological, and/or emotional events or states.

In some embodiments, examples of earring(s) described herein can reliably monitor the core body temperature by measuring the temperature of their external ear(s) and/or the ambient temperature, simultaneously, continually (e.g., in real time, or in near real time), or in time intervals.

FIG. 1 is a schematic illustration of a system 100 arranged in accordance with examples described herein. FIG. 1 depicts a user 102, an external ear 104 (e.g., an auricle of an ear, an outer ear) of the user 102, an earring 106 with a first portion 108 and a second portion 110, a coupling 112 between the first portion 108 and the second portion 110 of the earring 106, and a communication coupling 114 between the second portion 110 of the earring 106 and a computing system 116, in accordance with examples described herein. Alternatively (not illustrated as such in FIG. 1), in some embodiments, the communication coupling 114 may be between the first portion 108 of the earring 106 and the computing system 116. FIG. 1 may not illustrate all components of the earring 106. For example, the earring 106 may include a power source (e.g., a battery), a communication interface, an antenna, a post through an ear piercing, a clip to attach the earring to the ear, and/or other components.

In some embodiments, the computing system 116 may include or utilize a processor(s) 118; a computer readable media 120 encoded with instructions for reading sensor data 122, instructions for analyzing external ear temperature 124 (e.g., earlobe temperature of the user 102), instructions for analyzing ambient temperature 126, and/or instructions for determining physiological, psychological, and/or emotional events 128; a display 130; a speaker 132; an additional computer readable media 134 storing, or utilized by, application(s) 136; and communication interface(s) 138. In some embodiments, the computing system 116 may include fewer, additional, and/or different components than what is shown in FIG. 1.

Examples of the computing system 116 may include a smartphone, a tablet, a laptop, a desktop computer, a smartwatch, computing or smart eyeglasses, a VR/AR headset, a gaming system or controller, a smart speaker system, a television, an entertainment system, an automobile or a function thereof, a trackpad, a drawing pad, a netbook, an e-reader, a home security system, an appliance, and/or other computing systems.

The anatomical features of an external ear 104 play roles in directing sound waves into the ear canal. Anatomically, the external ear 104, includes: an helix, which is the outer rim forming the curved ridge; an antihelix, which is a curved prominence of cartilage inside the helix; a tragus, which is a relatively small pointed eminence in front of the ear canal; an antitragus, which is a small tubercle opposite the tragus; a concha, which is the hollow next to the ear canal leading to the auditory canal; a lobule (e.g., an earlobe), which is the lower fleshy part of the ear; a scapha, which is the groove between the helix and antihelix; and a fossa triangularis (e.g., a triangular fossa), which is a depression between the two crura of the antihelix.

FIG. 1 shows a dangling earring 106 design, where: the first portion 108 of the earring 106 is abutted to the earlobe of the external ear 104; and the second portion 110 of the earring 106 is dangling, and the second portion 110 of the earring 106 is not in direct contact with the external ear 104 or any other part of the body of the user. The earlobe of the user may be pierced or unpierced. In cases where the earlobe is pierced, the first portion 108 of the earring 106 may include or utilize a butterfly back, a post backs, a screw back, a lever back, a fishhook back, a latch back, or another fastening mechanism that utilizes a pierced ear. In cases where the earlobe is unpierced, the first portion 108 of the earring 106 may be a clip-on earring, a magnetic earring, an ear cuff, a stick-on earring, a spring-loaded earring, or another type of earring that does not require pierced ears.

Although not illustrated as such in FIG. 1, in some embodiments, the first portion 108 of the earring 106 may be abutted on another portion of the external ear 104, such as the helix, the antihelix, etc. Nevertheless, the second portion 110 of the earring 106 is still dangling and not abutted to the external ear 104 or another portion of the body. The length of the coupling 112 between the first portion 108 and the second portion 110 may be long enough to accommodate the dangling of the second portion 110, such that the second portion 110 of the earring 106 is not abutted to the external ear 104 or another portion of the body of the user (e.g., the user 102).

Examples of software described herein may include instructions to analyze external ear temperature and/or instructions to analyze ambient temperature, such as the instructions for analyzing external ear temperature 124 and/or the instructions for analyzing ambient temperature 126 of FIG. 1. The instructions to analyze external ear temperature may receive the external ear temperature from sensors described herein and may manipulate or otherwise analyze that data. For example, the instructions may include instructions to calculate changes in the external ear temperature over time, to find a maximum external ear temperature over a period, a minimum external ear temperature over a period, a rate of change of external ear temperature, or other combinations of external ear temperature sensor data. As other examples, the instructions may include instructions to repeat, remove, and/or ignore external ear temperature data.

The instructions to analyze ambient temperature may receive the ambient temperature from sensors described herein (e.g., dangling sensor(s)) and may manipulate or otherwise analyze that data. For example, the instructions may include instructions to calculate changes in the ambient temperature over time, to find a maximum ambient temperature over a period, a minimum ambient temperature over a period, a rate of change of ambient temperature, or other combinations of ambient temperature sensor data. As other examples, the instructions may include instructions to repeat, remove, and/or ignore ambient temperature data.

In some embodiments, the first portion 108 of the earring 106 may include a first temperature sensor, and the second portion 110 of the earring 106 may include a second temperature sensor. The first temperature sensor may be configured to detect and/or measure an earlobe temperature at an external ear 104 (e.g., the earlobe of user 102), and the second temperature sensor may be configured to detect and/or measure an ambient temperature. Therefore, the earring 106 includes or utilizes a dual temperature sensor design. Accordingly, in examples described herein, wearable devices may include two temperature sensors. A first temperature sensor may be positioned to measure an external ear 104 temperature, and/or the temperature of the earlobe of the user 102. A second temperature sensor may be positioned to measure an ambient temperature. The second temperature sensor may be distanced from the body of the user so as to obtain the ambient temperature measurement rather than the body temperature, the skin temperature, the temperature of the external ear 104, or the earlobe temperature.

In some embodiments, the user 102 may wear and/or utilize one or more earrings 106, such as a first earring on an auricle of an ear, a second earring on the other auricle of the other ear, two earrings on the same auricle, two earrings on each auricle of each ear, and other combinations. Therefore, FIG. 1 does not depict all combinations. In some embodiments, when the user 102 wears and/or utilizes more than one earring, the external ear 104's (e.g., earlobe of the external ear 104) temperature and/or the ambient temperature may be measured using a redundant temperature measurement.

In some embodiments, assume the user 102 wears a first earring on an auricle of an ear (e.g., the left ear), and a second earring on an external ear of the other ear (e.g., the right ear). In such a case, the temperature sensor embedded in or on the first portion 108 of the first earring 106 (e.g., earring on the left ear) measures the external ear temperature; the temperature sensor embedded in or on the second portion 110 of the first earring 106 (e.g., earring on the left ear) measures the ambient temperature; the temperature sensor embedded in or on first portion 108 of the second earring 106 (e.g., earring on the right ear) measures the external ear temperature; and the temperature sensor embedded in or on second portion 110 of the second earring 106 (e.g., earring on the right ear) measures the ambient temperature. Normally, the temperature measurements of both external ears (e.g., both earlobes) using both earrings should be the same or nearly the same, and the ambient temperature measurements using both earrings should be the same or nearly the same. However, should the external ears' temperature measurements differ, the system 100 can repeat the temperature measurements, highlight and calculate the difference between the temperature measurements of the two external ears, ignore one of the external ears' temperature measurements, ignore both external ears' temperature measurements, or combinations thereof. Similarly, should the ambient temperature measurements differ, the system 100 can repeat the temperature measurements, highlight and/or calculate the difference between the two ambient temperature measurements, ignore one ambient temperature measurements, ignore both ambient temperature measurements, or combinations thereof. For example, assume the user 102 is driving with her car window rolled down. In some cases, the ambient temperature near her left ear may differ from the ambient temperature of her right ear. In such a case, the computing system 102 may ignore one or more of the ambient temperatures measured by one of the user's earrings (e.g., earring 106).

In some embodiments, the processor(s) 118 may be implemented using an electronic device that may be capable of processing, receiving, and/or transmitting instructions that may be included in, permanently or temporarily saved on, and/or accessed by computer readable media 120, the additional computer readable media 134, or another computer readable media that is not illustrated in FIG. 1. In aspects, the processor(s) 118 may be implemented using one or more processors (e.g., a central processing unit (CPU), a graphic processing unit (GPU)), and/or other circuitry, where the other circuitry may include at least one or more of an application specific integrated circuit (ASIC), a field programmable gate array (FPGA), a microprocessor, a microcomputer, and/or the like. Furthermore, the processor(s) 118 may be configured to execute the instructions for reading sensor data 122; the instructions for analyzing an external ear temperature 124 (e.g., the temperature of the external ear 104, the temperature of the earlobe of the user 102); the instructions for analyzing ambient temperature 126; the instructions for determining physiological, psychological, and/or emotional events 128; the instructions associated with application(s) 136; or other instructions, in parallel, locally, and/or across a network, for example, by using cloud and/or server computing resources.

In some embodiments, the processor(s) 118 may execute instructions to operate, associated with, and/or part of, the computer readable media 120; the instructions for reading sensor data 122; the instructions for analyzing an external ear temperature 124; the instructions for analyzing ambient temperature 126; the instructions for determining physiological, psychological, and/or emotional events 128; display 130; the speaker 132; the additional computer readable media 134; the application(s) 136; the communication interface(s) 138; and/or other components and/or entities of, or coupled with, the computing system 116 that may not be explicitly illustrated in FIG. 1.

The instructions for determining events, such as the instructions for determining physiological, psychological, and/or emotional events 128, may utilize the external ear temperature and ambient temperature measurements received and/or calculated by the computing system 116. For example, A change in external ear temperature data received at the computing system 116 may be compared with a change in ambient temperature received at the computing system 116. In this manner, the instructions for determining events may determine an event based on a change in external ear temperature, adjusted for a change in ambient temperature.

In some embodiments, the computer readable media 120 and/or the additional computer readable media 134 may be and/or include any data storage media, such as volatile memory and/or non-volatile memory. Examples of volatile memory may include a random-access memory (RAM), such as a static RAM (SRAM), a dynamic RAM (DRAM), or a combination thereof. Examples of non-volatile memory may include a read-only memory (ROM), a flash memory (e.g., NAND flash memory, NOR flash memory), a magnetic storage medium, an optical medium, a ferroelectric RAM (FeRAM), a resistive RAM (RRAM), and so forth.

In some embodiments, the display 130 may utilize a variety of display technologies, such as a liquid-crystal display (LCD) technology, a light-emitting diode (LED) backlit LCD technology, a thin-film transistor (TFT) LCD technology, an LED display technology, an organic LED (OLED) display technology, an active-matrix OLED (AMOLED) display technology, a super AMOLED display technology, and so forth.

In some embodiments, the instructions for reading sensor data 122, the instructions for analyzing an external ear temperature 124, the instructions for analyzing ambient temperature 126, and/or the instructions for determining physiological, psychological, and/or emotional events 128 may be included in, permanently or temporarily saved on, and/or accessed by the computer readable media 120 of the computing system 116. These instructions my include code, pseudo-code, algorithms, a machine learning model, heuristics, other models, software, and/or so forth and may be executable by the processor(s) 118.

FIG. 1 shows that the application(s) 136 is stored in the additional computer readable media 134. Alternatively, the application(s) 136 may be stored in the computer readable media 120 or another computer readable media. Alternatively, the application(s) 136 or portions of the application(s) 136 may be stored on a server (e.g., a cloud server), and the user may utilize the computing system 116 to access and utilize the application(s) 136.

In some embodiments, the application(s) 136 may be a software application installed on the computing system 116 or accessed using the computing system 116; a function of the computing system 116; a peripheral of the computing system 116; or another entity.

In some embodiments, the application(s) 136 may use or access the instructions for reading sensor data 122, the instructions for analyzing an external ear temperature 124, the instructions for analyzing ambient temperature 126, and/or the instructions for instructions for determining physiological, psychological, and/or emotional events 128.

In some embodiments, the application(s) 136 may support or include a graphical user interface (GUI) that aids the user 102 to interact with computing system 116 and/or the application(s) 136 through, for example, graphical icons, visual indicators, windows, buttons, menus, etc. The GUI(s) of the application(s) 136 may be designed to be intuitive and/or user-friendly. The GUI(s) can provide visual representations of functions; temperature measurements of the external ear 104; monitor changes of the core body temperature; temperature measurements of the ambient; a determination or classification of a physiological, psychological, and/or emotional event; instructions to the user 102; and/or other actions.

In some embodiments, the user 102 may use the application(s) 136, and, based on the user's external ear (e.g., auricle, earlobe) temperature, the system 100 may determine that the user 102 is experiencing stress. In such a case, the application(s) 136 may display on the display 130 instructions to the user 102, such as “take five deep breaths;” “meditate;” “take a short walk;” and/or other instructions that medical researchers have found to be helpful in stress management. Additionally, or alternatively, the application(s) 136 may use the speaker 132 to read aloud the instructions to the user 102.

In some embodiments, the application(s) 136 can collect the temperature data from the earring 106 using a communication protocol and/or standard. For a wireless communication, the application(s) 136 and/or the computing system 116 may continuously scan for the presence of the earring 106. For example, the earring 106 may include a device name, such as “Jane Doe's earring.” As another example, the application(s) 136 and/or the computing system 116 may collect the data seamlessly, without prompting the user 102 to start collect temperature data. It is to be understood, however, that the application(s) 136 and/or computing system 116 give(s) the user 102 full control on when to start collecting the temperature data and what happens to the temperature data. For example, the user 102 may want to share the temperature data with their physician, sports trainer, etc. In such situations, the user 102 makes the decision whether they want to share the temperature data or not share the temperature data. As another example, the user 102 may configure the application(s) 136 and/or the computing system 116 to permanently delete the temperature data automatically, for example, after a time period has passed (e.g., every day, every week, every month, or another time period).

In some embodiments, the earring 106, the computing system 116, the application(s) 136, and/or any other system(s) and/or method(s) described herein may meet or exceed industry best-practices. For example, in the USA, if these systems and/or methods are used in the medical field, they may meet and/or exceed the medical standards established by the Health Insurance Portability and Accountability Act of 1996 (HIPAA). As another example, internationally, if these systems and/or methods are used in sports training and/or sports medicine, they may meet or exceed the standards set by, for example, the Fédération Internationale de Football Association (FIFA) and/or any other international sports association. Therefore, depending on the application, these systems and/or methods may meet and/or exceed any standards established and/or adopted in local, state, country, and/or international jurisdictions.

In some embodiments, the application(s) 136 and/or the computing system 116 can collect the temperature data from the earring 106 in real-time or in time intervals (e.g., every minute, hour, day, etc.). In some embodiments, the temperature data may be displayed on the display 130 when the user uses the application(s) 136. In some embodiments, the application(s) 136 may create a graph(s) that may plot the temperature changes over a time period (e.g., a ten-minute time period, a 30-minute time period, a one-hour time period, a 24-hour time period, a week time period, etc.). In some embodiments, the collected temperature data may be saved to one or more local storage devices and/or computer readable media on the computing system 116 or on a server for further analysis. In some embodiments, the application(s) 136 can be a valuable tool for the user 102 to understand their physiological, psychological, and/or emotional events or states. In some embodiments, the user 102 can then make physical and/or behavioral changes, should they decide to manage their physiological, psychological, and/or emotional events or states.

In some embodiments, the communication interface(s) 138 of the computing system 116 and/or a communication interface (not shown in FIG. 1) of the earring 106 may be configured to receive and/or transmit between said entities, for example, by using the communication coupling 114. Alternatively, or additionally, the computing system 116 and the earring 106 may utilize their respective communication interfaces to communicate with each other indirectly by, for example, using a network (not illustrated in FIG. 1). In some embodiments, each or either of the communication interfaces may communicate with a server (not illustrated in FIG. 1), for example, via the network. In some embodiments, the communication interface(s) 138 of the computing system 116 and/or the communication interface of the earring 106 may include and/or utilize an application programming interface (API) that may interface and/or translate requests across the earring 106, the computing system 116, the computer readable media 120, the additional computer readable media 134, the network, and/or the server.

FIG. 1 illustrates that the earring 106 communicates temperature sensor data to the computing system 116 wirelessly, for example, using a wireless communication coupling 114. The wireless communication coupling 114 may be user friendly, since the user 102 need not plug in communication cables to transmit data from the earring 106 to the computing system 116. Alternatively, however, the earring 106 may communicate temperature sensor data to the computing system 116 using a wired communication coupling. Therefore, the communication interface of the earring 106, the communication interface(s) 138 of the computing system 116, and/or the network may support a wired and/or a wireless communication using a variety of communication protocols and/or standards. Examples of such protocols and standards include: a 3rd Generation Partnership Project (3GPP) Long-Term Evolution (LTE) standard, such as a 4th Generation (4G) or a 5th Generation (5G) cellular standard; an Institute of Electrical and Electronics (IEEE) 602.11 standard, such as IEEE 602.11g, ac, ax, ad, aj, or ay (e.g., Wi-Fi 6® or WiGig®); an IEEE 602.16 standard (e.g., WiMAX®); a Bluetooth Classic® standard; a Bluetooth Low Energy® or BLE® standard; an IEEE 602.15.4 standard (e.g., Thread® or ZigBee®); other protocols and/or standards that may be established and/or maintained by various governmental, industry, and/or academia consortiums, organizations, and/or agencies; and so forth.

If the earring 106 and the computing system 116 communicate using a network (not illustrated as such in FIG. 1), the network may be a cellular network, the Internet, a wide area network (WAN), a local area network (LAN), a wireless LAN (WLAN), a wireless personal-area-network (WPAN), a mesh network, a wireless wide area network (WWAN), a peer-to-peer (P2P) network, and/or a Global Navigation Satellite System (GNSS) (e.g., Global Positioning System (GPS), Galileo, Quasi-Zenith Satellite System (QZSS), BeiDou, GLObal NAvigation Satellite System (GLONASS), Indian Regional Navigation Satellite System (IRNSS), and so forth).

In addition to, or alternatively of, the communications illustrated in FIG. 1, the earring 106, the computing system 116, or another entity (e.g., a server) may facilitate other unidirectional, bidirectional, wired, wireless, direct, and/or indirect communications utilizing one or more communication protocols and/or standards. Therefore, FIG. 1 does not necessarily illustrate all communication signals or communication couplings that may be used in various examples.

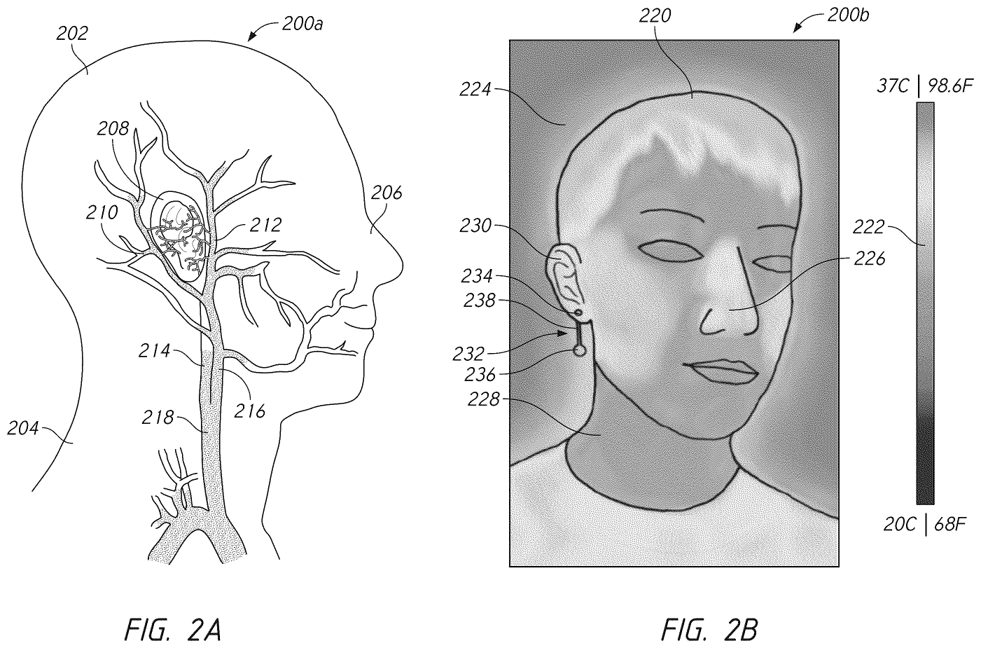

FIG. 2A is a schematic illustration 200a of blood vessels around an external ear of a user, in accordance with examples described herein. FIG. 2B is schematic illustration 200b of heat distribution around an external ear, a head, and/or a neck of a user, and/or an ambient heat distribution, in accordance with examples described herein.

FIG. 2A and/or the schematic illustration 200a shows a head 202, a neck 204, a nose 206, an external ear 208, a posterior auricular artery 210, a superficial temporal artery 212, an internal carotid artery 214, an external carotid artery 216, and a common carotid artery 218 of a user.

FIG. 2B and/or the schematic illustration 200b shows a head 220 temperature, a temperature spectrum 222, an ambient 224 temperature, a nose 226 temperature, a neck 228 temperature, an external ear 230 temperature, an earring 232, a first portion 234 of the earring 232, a second portion 236 of the earring 232, and a coupling 238 between the first portion 234 and the second portion 236 of the earring 232. In some embodiments, a user may utilize the earring 232 to detect and/or monitor changes into their physiological, psychological, and/or emotional state by detecting and/or monitoring temperature changes of the external ear 230 and/or the ambient 224. In some embodiments, the first portion 234 of the earring 232 can detect and/or monitor temperature changes of the external ear 230, such as the earlobe of the external ear 230. Meanwhile, the second portion 236 of the earring 232 can detect and/or monitor temperature changes of the ambient 224.

The head and earring shown in FIGS. 2A and 2B may be used to implement and/or may be implemented by components shown in FIG. 1. For example, the head 202 and/or head 220 may be the head of the user 102 of FIG. 1. The earring 232 may be used to implement and/or may be implemented by the earring 106 of FIG. 1.

Generally, core body temperature may refer to the internal body temperature, for example, of the tissues and organs of a person (e.g., a user, a patient). The core body temperature can be regulated by the body's thermoregulatory system, which ensures relative stability of the core body temperature, despite external temperature changes. The average core body temperature for a healthy person is around 37° C. (or 98.6° F.).

In some embodiments, some physiological states, diseases, and/or conditions can cause a drop in core body temperature, known as hypothermia. These physiological states may include: hypothyroidism, which may be caused by insufficient thyroid hormone production, leading to decreased metabolic rate and heat production; sepsis, which may be caused by severe infections causing systemic inflammation and impaired thermoregulation; hypoglycemia, which may be caused by low blood sugar levels reducing energy availability for heat production; malnutrition, which may impair metabolic processes and/or thermoregulation; adrenal insufficiency, which may affect metabolism and/or heat production; extensive burns, where severe skin damage may disrupt thermal regulation and heat retention; a combination of the aforementioned diseases and/or conditions; and/or other diseases and/or conditions.

In some embodiments, some physiological states, diseases, and/or conditions can cause an increase in core body temperature, known as hyperthermia or fever. These physiological states may include: infections, such as bacterial, viral, and fungal infections (e.g., influenza, tuberculosis, and sepsis); hyperthyroidism, which may lead to increased metabolic rate and heat production; heat stroke, which may be caused by exposure to high temperatures and/or inadequate cooling; autoimmune diseases, such as systemic lupus erythematosus and rheumatoid arthritis, which can cause inflammatory responses that elevate core body temperature; malignant hyperthermia; endocrine disorders; a combination of the aforementioned diseases and/or conditions; and/or other diseases and/or conditions.

In some embodiments, some physiological, psychological, and/or emotional events or states can cause a change of the external ear (e.g., earlobe) temperature and/or a change in the core body temperature when a user 102 is eating, exercising, feeling stress, feeling excitement, meditating, praying, sleeping, ovulating, experiencing vasomotor symptoms (VMS), or combinations thereof.

In some embodiments, skin temperature may differ from core body temperature. For example, while the core body temperature may remain at 37° C. (98.6° F.), skin temperature may be lower or higher than the core body temperature. As another example, skin temperature may fluctuate across body regions, but the core body temperature may remain stable, or nearly stable. Generally, the skin plays a vital role in thermoregulation through insulation, blood flow control (e.g., vasodilation, vasoconstriction), and sweating. Vasodilation may increase blood flow to the skin, and vasodilation may aid with heat dissipation. Vasoconstriction may reduce blood flow to the skin, and vasoconstriction may conserve the core body temperature. Sweating may facilitate heat evaporation and cooling of the skin. Overall, the skin may respond to internal and/or external temperature changes to keep the core body temperature stable, or nearly stable. In some embodiments, skin temperature changes may indicate stress, with elevated temperatures in facial and forehead regions during stress, and decreased skin temperature in finger(s) and the nose region of a person. As a result, monitoring skin temperature changes at an external ear can offer valuable insight(s) into a person's physiological, psychological, and/or emotional state.

The human external ear is one of the acral regions that can help regulate core body temperature. For example, the earlobe of the external ear 230 has a large blood supply that aids in maintaining temperature balance by controlling blood vessels. As is shown in FIG. 2A and/or the schematic illustration 200a, the posterior auricular artery 210 supplies blood to the back of the ear, while the superficial temporal artery 212 supplies blood to the front of the ear, face, and certain areas of the head 202. The posterior auricular artery 210 and the superficial temporal artery 212 stem from the external carotid artery 216 in the neck 204. In some embodiments, the posterior auricular artery 210 and the superficial temporal artery 212 can contribute to the regulation of ear temperature. Given its proximity to the head and minimal susceptibility to motion artifacts of the user, the external ear 230 (and the earlobe of the external ear 230) serves as an advantageous location for corporeal temperature (or core body temperature) monitoring.

In some embodiments, the earlobe's close proximity to the brain can aid in capturing changes related to psychological and/or emotional states of the user (e.g., stress and/or emotions), in addition to, or alternatively of, physiological states (e.g., illness, disease). For example, emotional changes, such as embarrassment, anxiety, anger, etc. can cause the ears and face to “turn red,” due to the autonomic nervous system's response. The person's emotional response may involve dilated blood vessels and increased blood flow from the superficial temporal artery 212 that may be triggered by the release of adrenaline, which may lead to a rise in temperature in the external ear 208, face, etc. Consequently, the temperature of the external ear 208 (e.g., the earlobe temperature) can serve as a valuable biomarker for detecting and/or monitoring changes in physiological, psychological, and/or emotional states.

In some embodiments, to distinguish changes in the earring temperature signal due to changes of the core body temperature from changes caused by the ambient temperature, the second portion 236 of the earring 232 can increase the isolation of the ambient temperature from the core body temperature and/or the external ear 230 temperature. The dangling second portion 236 of the earring 232 can detect the ambient temperature around the ear, rather than the core body temperature or the external ear 230 temperature itself. In practice, the temperature sensor embedded in or on the second portion 236 of the earring 232 may still couple to the heat from the body of the user because the ambient temperature (e.g., air temperature) around the ear may be affected by the radiated heat from, for example, the neck 228 and the head 220. However, the radiated heat from the neck 228 and/or the head 220 to the second portion 236 of the earring 232 may be relatively low, or in some instances, negligeable. Therefore, the temperature sensor embedded in or on the second portion 236 of the earring 232 provides valuable information for detecting changes in ambient temperature. Meanwhile, the temperature sensor embedded in or on the first portion 234 of the earring 232 provides valuable information corporeal temperature, core body temperature, and/or external ear 230 temperature (e.g., earlobe temperature).

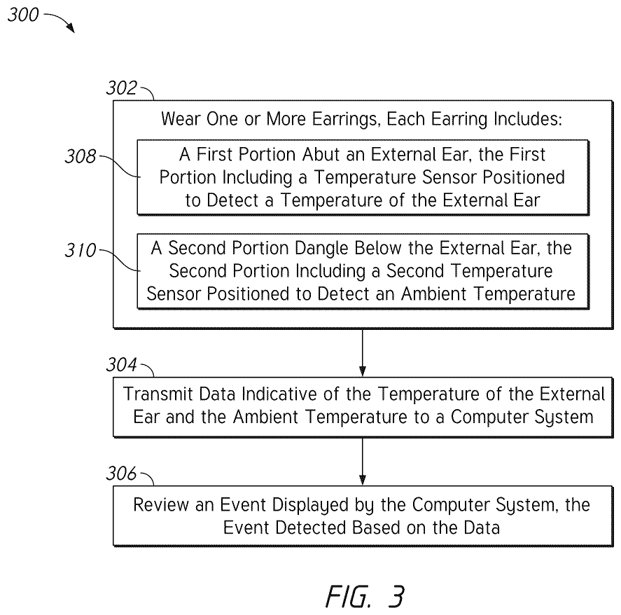

FIG. 3 is a flowchart of a method 300 arranged in accordance with examples described herein. In some embodiments, block 302 of the method 300 includes a user wearing one or more earrings. For example, the user 102 of FIG. 1 may wear one or more earrings 106 of FIG. 1. The method may be performed, for example, by the user 102 of FIG. 1.

In some embodiments, each earring includes, in block 308 of the method 300, a first portion of the earring abutted an external ear (e.g., earlobe) of the user, where the first portion includes a temperature sensor positioned to detect a temperature of the external ear (e.g., the earlobe). For example, the temperature sensor embedded in or on the first portion 108 of the earring 106 of FIG. 1 may detect the temperature of the external ear (e.g., the earlobe) of the user. As described herein, the external ear temperature is positively correlated (e.g., instead of inversely correlated) with the core body temperature.

In some embodiments, each earring includes, in block 310 of the method 300, a second portion of the earring, where the second portion dangles below the external ear, and where the second portion includes a second temperature sensor positioned to detect an ambient temperature. For example, the temperature sensor embedded in or on the second portion 110 of the earring 106 of FIG. 1 may detect the ambient temperature.

In some embodiments, each earring transmits, in block 304 of the method 300, data indicative of the temperature of the external ear (e.g., earlobe) and the ambient temperature to a computer system. For example, the earring 106 of FIG. 1 transmits the temperature data of the external ear and the ambient to the computing system 116 of FIG. 1 using a communication coupling 114 of FIG. 1.

In some embodiments, the user, in block 306 of method 300, reviews an event displayed by the computer system, the event detected based on the data. For example, the event includes the user eating; exercising; experiencing stress; experiencing excitement; meditating; praying; sleeping; ovulating; experiencing vasomotor symptoms (VMS); having hypothyroidism; having sepsis; having hypoglycemia; experiencing the effects of malnutrition; experiencing the effects adrenal insufficiency; having extensive burns; having an infection; having hyperthyroidism; having an autoimmune disease; having malignant hyperthermia; having an endocrine disorder; or combinations thereof.

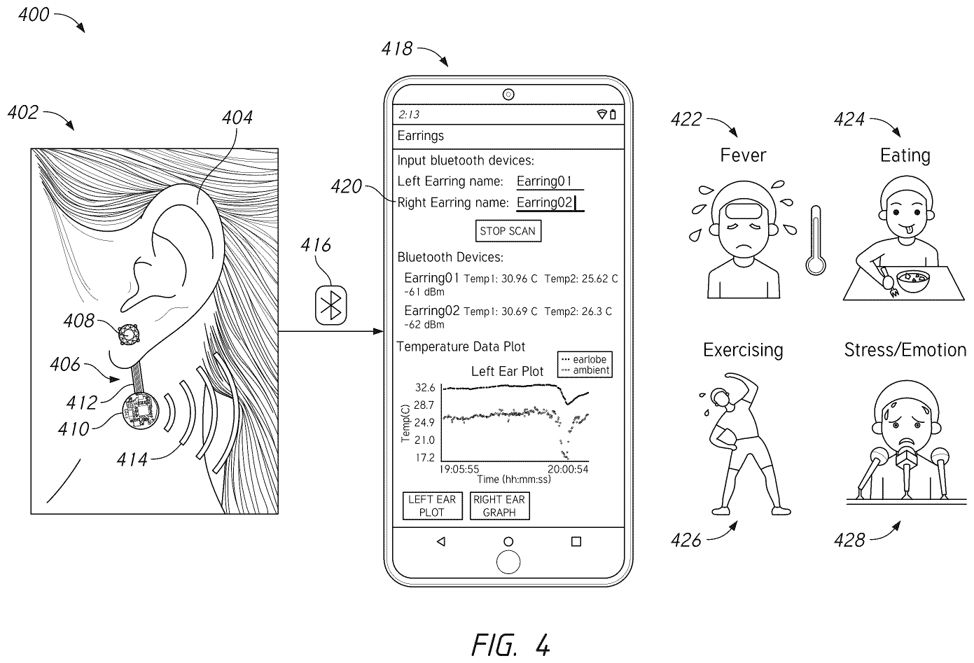

FIG. 4 is a schematic illustration of a system 400 arranged in accordance with examples described herein. In some embodiments, the system 400 of FIG. 4 may be an example(s) implementation(s) of the system 100 of FIG. 1.

FIG. 4 depicts a user 402; an external ear 404 of the user 402; an earring 406 with a first portion 408 and a second portion 410, and a coupling 412 between the first portion 408 and the second portion 410 of the earring 406; a communication coupling 414 using a Bluetooth 416 protocol and/or standard (e.g., Bluetooth Classic®, a Bluetooth Low Energy® or BLE®); a smartphone 418; an application 420 used to determine and/or monitor physiological, psychological, and/or emotional events or states; an event 422; an event 424; an event 426; and an event 428.

FIG. 4 illustrates four events, where the event 422 illustrates the user 402 having a fever; the event 424 illustrates the user 402 eating; the event 426 illustrates the user 402 exercising; and the event 428 illustrates the user experiencing stress or emotions. Although not illustrated in FIG. 4, the system 400 can be used to detect and/or monitor other physiological states, psychological states, and/or emotional states. Generally, the system 400 can be used to detect and/or monitor eating, exercising, stress, excitement, meditating, praying, sleeping, ovulating, vasomotor symptoms (VMS), hypothyroidism, sepsis, hypoglycemia, malnutrition, adrenal insufficiency, extensive burns, an infection, hyperthyroidism, an autoimmune disease, malignant hyperthermia, an endocrine disorder, or combinations thereof.

FIG. 4 depicts a smartphone which may be used to detect and display events described herein.

Note that the earring 406 of FIG. 4 may be used to implement and/or may be implemented by the earring 106 of FIG. 1. The smartphone 418 of FIG. 4 may be used to implement and/or may be implemented by the computing system 116 of FIG. 1. The application 420 implemented on the smartphone 418 of FIG. 4 may be implemented using some or all of the executable instructions shown in FIG. 1, such as the executable instructions for determining physiological, psychological, and/or emotional events 128. Accordingly, the events shown in FIG. 4 may be detected based on ambient and external ear 404 (e.g., earlobe) temperature measurements received from an earring.

In some embodiments, the earring 406 can utilize the unique position of the earring 406 to the proximity to the head. The head of the users 402 has tight temperature coupling to the core body temperature. Therefore, the earring 406 can monitor changes of the core body temperature by measuring the external ear temperature better than other wearable devices, such as smartwatches or other wearable devices, which may be worn on extremities of a user.

In some embodiments, the earring 406 has a dual temperature sensor design, where a first temperature sensor is embedded in or on the first portion 408 of the earring 406, and a second temperature sensor is embedded in or on the second portion 410 of the earring 406. Therefore, the earring 406 can differentiate the external ear and/or the core body temperature change(s) of the user 402 from ambient temperature change(s).

In some embodiments, the earlobe temperatures may be stable within, for example, ±0.32° C. when a user is resting. In some embodiments, the temperature of the external ear (e.g., earlobe) may change when a user has a fever, eats, exercises, and/or is experiencing stress or emotion. For example, when the user 402 experiences certain stressful situations, such as public speaking, taking exams, working on school projects, etc., the earring(s) may measure changes in earlobe temperature. The external ear (e.g., earlobe) temperature may be correlated to the core body temperature. In some embodiments, the system 400 can differentiate and/or categorize the activities based on temperature changes of the earlobes of the subject(s) (e.g., a user participating in an experiment) and/or the user(s). Therefore, the system 400 can be used to determine physiological, psychological, and/or emotional events of the user 402.

In some embodiments, when the user 402 is eating (e.g., event 424), the temperature of external ear 404 (e.g., the earlobe) may rise due to factors, such as an increased metabolism during digestion, food temperature, and chewing movements. The system 400, the earring 406, the smartphone 418, and/or the application 420 can detect that event 424 and can differentiate the event 424 from other events (e.g., event 422, event 426, event 428, etc.).

In some embodiments, the external ear 404 temperature of the user 402 changes (e.g., increases) during stressful events, such as public speaking (e.g., event 428). This temperature increase may be caused by the blood flow change within the superficial temporal artery 212 of FIG. 2A and the posterior auricular artery 210 of FIG. 2A during stressful events. While stress has been investigated using other physiological biomarkers, such as like heart rate, heart rate variability, blood pressure, and/or skin conductance, these metrics often struggle to differentiate between various types of events, such as event 422, event 424, event 426, event 428, etc. For example, other biomarkers may struggle to differentiate stress (e.g., a first event) from exercising (e.g., a second event) solely based on an elevated heart rate. However, the system 400 (e.g., earring 406, smartphone 418, application 420) effectively differentiates between event 422, event 424, event 426, event 428, and/or other events that are not illustrated in FIG. 4.

In some embodiments, the application 420 can collect the temperature data from the earring 406 using Bluetooth 416. When the user 402 opens or uses the application 420, the smartphone 418 may scan (or search) for Bluetooth devices that are filtered by the earring 406's specific Bluetooth name (e.g., Jane Doe's earring), enabling seamless and convenient temperature data collection. In some embodiments, the application 420 may display (e.g., on the display 130 of FIG. 1) the temperature data in real time, near real time, or in time intervals. In some embodiments, the temperature data may be displayed when the user 402 uses the application 420, along with a graph that plots the temperature changes over a time period, such as a one-minute, ten-minute, 30-minute, etc. period. Therefore, in some embodiments, when the user 402 uses the application 420, the smartphone 418 may display, the event (e.g., event 422, event 424, event 426, or event 428), instructions to a user, the external ear 404 temperature, the ambient temperature, a first graph of the external ear temperature over time, a second graph of the ambient temperature over time, other graphs, or combinations thereof.

Fever is a common physiological response to a variety of medical conditions. Clinically, fever may be defined as an elevated core body temperature exceeding 37.8° C. (100° F.) when measured orally. An example of the system 400 was used to conduct an experiment aimed to investigate the effects of fever on earlobe temperature and explore the feasibility of using the earring 406 to detect and monitor fever (e.g., event 422 of FIG. 4). In an implemented study, 25 participants were recruited, including: four (4) febrile individuals with a core body temperature higher than 37.8° C. (100° F.); one (1) individual (female) with a slightly elevated core body temperature of 37.6° C. (99.7° F.) in close proximity to fever; and 20 healthy individuals with a core body temperature around 37° C. (98.6° F.). During the data collection process, each participant wore the earring 406 for a duration of five (5) minutes to obtain earlobe temperature readings. Additionally, their oral temperature was measured, while wearing the earring 406. The measurements were conducted in a similar room environment with a temperature ranging from 20° C. to 22° C. (68° F. to 71.6° F.). The febrile participants had an average earlobe temperature of 35.62±1.8° C. (96.12±3.24° F.), which is significantly higher than the healthy participants' average earlobe temperature of 29.7±0.74° C. (85.5±1.33° F.).

The study revealed that, in general, healthy female participants have a higher earlobe temperature of 30.11±0.78° C. (86.2±1.4° F.) compared to healthy male participants' average earlobe temperature of 29.26±0.70° C. (84.67±1.26° F.). Therefore, in some embodiments, when the user 402 configures the application 420, the application 420 may prompt the user to identify their gender, in order to increase the accuracy of detecting and/or determining physiological, psychological, and/or emotional events.

In the implemented study, the lowest temperature among febrile patients was observed in older (e.g., age) study participants. Therefore, in some embodiments, when the user 402 configures the application 420, the application 420 may prompt the user to insert their age, in order to increase the accuracy of detecting and/or determining physiological, psychological, and/or emotional events.

Computing systems described herein may utilize patient data, such as gender, in addition to the ambient and external ear temperature measurements, to detect events. For example, the instructions for determining physiological, psychological, and/or emotional events 128 of FIG. 1 may include instructions for determining an event based on ambient temperature and external ear temperature received from an earring, combined with patient data such as gender, age, and/or weight.

In some embodiments, when the user 402 uses the application 420, the temperature of the external ear 404 (e.g., earlobe) may remain stable over time and may not change significantly over a short period of time (e.g., every second, every five seconds). In contrast, the dangling temperature sensor (e.g., the temperature sensor embedded in or on the second portion 410) may detect rapid changes due to the user's movements, causing airflow around the external ear 404. Additionally, or alternatively, there may be outlier temperature data points in, both, the external ear 404 and dangling temperature data, which may be caused by temperature sensor malfunction(s), wireless communication errors, or other errors. The application 420 can identify these outliers, since human temperature and ambient temperature does not change significantly in a considerable short period of time (e.g., in one second). In order to improve the interpretability of the dangling temperature data and eliminate outliers, in some embodiments, a moving-average filter with an empirical window length of, for example, 60 seconds may be applied to both temperature data (e.g., temperature measured by the first portion 408 of the earring 406, temperature measured by the second portion 410 of the earring 406).

In some embodiments, the application 420 may utilize a threshold-based heuristic technique for activity events detection using the combined temperature data from the first portion 408 and the second portion 410 of the earring 406. For example, the application 420 may execute instructions to identify periods when the environment ambient temperature is rapidly changing and may exclude these periods. To achieve this, the application 420 and/or the smartphone 418 may compute the temperature difference between the earlobe and dangling temperature, and use a threshold, of, for example, ±2° C. (±3.6° F.), on the computed temperature difference. This approach may suggest that the magnitude of the dangling temperature change exceeds that of the earlobe temperature during changes in ambient temperatures. The application 420 may, therefore, detect rapid ambient temperature changes, such as when the user 402 transitions from indoors to outdoors. In some embodiments, the application 420 can detect rapid ambient temperature change events occurring within a time period, such as within a five-minute, ten-minute, 15-minute, etc., time period. In some embodiments, the application 420 may exclude unstable environmental temperature within a time period to increase the accuracy of determining physiological, psychological, and/or emotional events. The application 420 and/or the smartphone 418 may do so by computing the temporal changes in external ear (e.g., earlobe) temperature using a time period (e.g., five, ten, 30 minutes, etc.) sliding window. For each window, the application 420 and/or the smartphone 418 may calculate the average external ear temperature during a previous time period, as the baseline temperature for the current time period window. By so doing, the application 420 and/or the smartphone 418 can account for changes in the room temperature or slow changes in the external ear temperature and/or the core body temperature. By subtracting the corresponding baseline from the current window's average external ear temperature, the application 420 and/or the smartphone 418 temperature delta.

In some embodiments, the application 420 may utilize a heuristic method on participants (e.g., willing participants of an experiment), utilizing a consistent window length(s) and/or threshold(s). Additionally, or alternatively, the application 420 can utilize pattern matching on a time-series temperature data. Additionally, or alternatively, the application 420 may utilize a machine learning classification model.

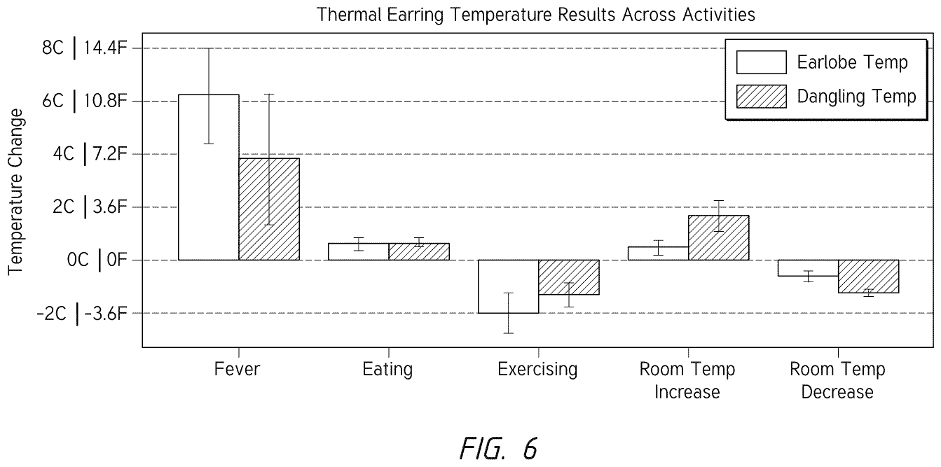

FIG. 6 is a schematic illustration of earring temperature results across activities arranged in accordance with examples described herein. The data shown in FIG. 6 may be received at one or more computing systems from temperature sensors included in one or more earrings described herein. In the example of FIG. 6, temperature changes over time are shown for both a external ear temperature (earlobe temperature of FIG. 6) and ambient temperature (dangling temperature). The temperature changes are shown for events including fever, eating, exercising, room temperature increase, and room temperature decrease.

During a fever event, for example, as shown in FIG. 6, the earlobe and/or the corporeal temperature may significantly increase, while ambient temperature may also increase, but to a lesser degree. During an eating event, both the earlobe and ambient temperature may show a minor increase. During an exercising event, the earlobe and/or the corporeal temperature may decrease to a greater extent than ambient temperature. During a room temperature increase, the earlobe and/or the corporeal temperature may increase a modest amount, while ambient temperature increases more significantly. During a room temperature decrease, the earlobe and/or the corporeal temperature may decrease a modest amount, while ambient temperature decreases more significantly.

Examples of computing systems described herein, such as the computing system 116 in accordance with the executable instructions for detecting events 128, may receive ambient and external ear temperature data from a wearable device. The computing system 116 may detect an event based on a comparison of the temperature data (e.g., both the earlobe and the ambient temperature data) with patterns associated with particular events, such as the patterns depicted in FIG. 6. Data indicative of patterns for particular events may be stored, for example, in memory accessible to the computing system 116 for use during execution of the instructions for detecting events.

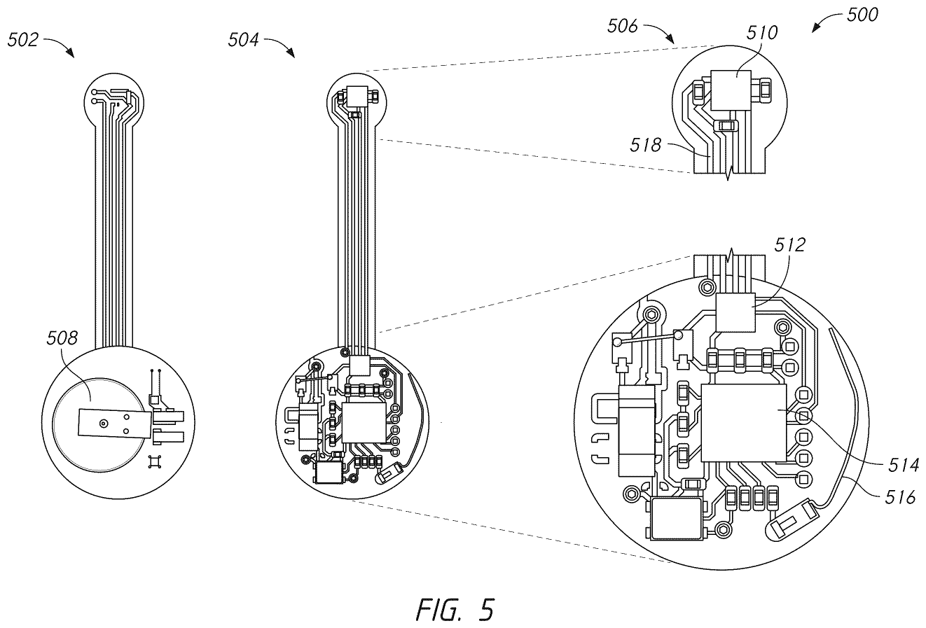

FIG. 5 is a schematic illustration of an earring 500 arranged in accordance with examples described herein. Specifically, FIG. 5 shows a first side view 502 (e.g., a backside view), a second side view 504 (e.g., a frontside view), and a zoomed-in view 506 of the second side view 504 of the earring 500.

In some embodiments, the earring 500 may include a battery 508, a first temperature sensor 510, a second temperature sensor 512, a wireless communication circuitry 514, an antenna 516, and a trace(s) 518 for electrically and/or communicatively coupling the various electronic components of the earring 500. FIG. 5 is an exemplary schematic illustration, and, in some embodiments, the earring 500 may include other electronic components, additional electronic components, or fewer electronic components.

In some embodiments, when designing the earring 500, the engineer and/or scientists may consider various constraints and/or parameters, such as size, weight, power consumption, communication (e.g., wireless communication) protocol and/or standard, and/or other constraints and/or parameter. In some embodiments, the user of the earring 500 may prefer an earring 500 that fits comfortably, is lightweight, and/or has a long or relatively long battery life to, for example, avoid frequent charging or battery replacement.

In some embodiments, the earring 500 includes a dual temperature sensor design that can sense the temperature of an external ear (e.g., an earlobe, an external ear 104) and the ambient air temperature, simultaneously. For example, the first temperature sensor 510 may be embedded in or on a first portion (e.g., first portion 108) of the earring 500, and the second temperature sensor 512 may be embedded in or on a second portion (e.g., second portion 110) of the earring 500. The earring 500, thereby, can differentiate the user's external ear and/or core body temperature changes from environmental and/or ambient temperature changes. The first temperature sensor 510 of the earring 500 is designed such that it is in direct (or near direct) contact with the external ear (e.g., the earlobe, the external ear 104) for sensing the earlobe temperature and/or the core body temperature. The second temperature sensor 512 of the earring 500 is embedded in or on the dangling part (e.g., the second portion 110 of FIG. 1) of the earring 500, and the second temperature sensor 512 can sense the ambient temperature, such as the temperature of the air around the external ear 104 of the user of the earring 500.

In some embodiments, the dangling temperature sensor (e.g., second temperature sensor 512 of the earring 500) may provide data to differentiate environmental changes, such as when a user walks from a temperature-controlled building to an outdoor environment, from the external ear temperature sensor (e.g., the first temperature sensor 510 of the earring 500), where the external ear temperature sensor can measure the external ear temperature and/or monitor changes of the core body temperature of the user wearing the one or more earrings 500.

Additionally, or alternatively, the earring 500 can be manufactured using another type of power source. For example, some users may find charging or replacing the battery 508 inconvenient. The power source, however, can harvest kinetic energy, leveraging the motion of the earring, while the earring is dangling when the user is moving. By harvesting kinetic energy from the earring's motion, a piezoelectric harvester can convert the vibrations of the dangling earring into electrical energy to power the earring. Additionally, or alternatively, the power source can harvest solar energy by harvesting energy, for example, from ambient light. A solar cell can provide power to charge the electronic components of the earring 500.

In some embodiments, the earring 500 of FIG. 5 may be built using a flexible printed circuit board (FPCB). The FPCB can allow the earring 500 to be flexible and allow movements of the dangling part of the earring 500 as the user moves their head or body, similar to traditional dangling earrings. The trace(s) 518 may be embedded in or on the FPCB.

### EXAMPLES

In some embodiments, the earring 500 may be manufactured in a laboratory using some off-the-shelf components. The earring 500 may have a width of approximately 11.3 mm, a length of approximately 31 mm, and a weight of approximately 335 mg. This example earring may consume approximately 14.4 μW and may a battery life of approximately 28 days. The example earring may be relatively small and light, and the earring 500 can be integrated into real jewelry with, for example, fashionable designs. The dimensions of the example earring may change. A manufacturer can use other electrical components to manufacture another earring, which may be smaller, larger, lighter, or heavier than the earring described herein. For example, a manufacturer that uses state-of-the-art manufacturing equipment may achieve better battery performance, may manufacture lighter earrings, etc.

In some embodiments, the example earring 500 may be built in a laboratory environment using an off-the-shelf temperature sensor, such as the HDC2010 produced by Texas Instruments. For example, the first temperature sensor 510 may be an HDC2010, and the second temperature sensor 512 may be another HDC2010. The size of the temperature sensor HDC2010 may be approximately 1.49 mm×1.49 mm, and the HDC2010 may consume approximately 0.9 μW of power. In some embodiments, the temperature sensor HDC2010 may measure the temperature with an accuracy within ±0.2° C. In some embodiments, the temperature sensor HDC2010 may draw 0.3 μA of average current, for example, when measuring temperature once per second. In some embodiments, the two temperature sensors may be connected to the wireless communication circuitry 514 through the same inter-integrated circuit (“I2C”), but with different I2C addresses for synchronized data acquisition.

In some embodiments, the earring 500 may communicate the sensed temperature data, energy levels of the power source (e.g., 10%, 50%, 90%, 100% of battery charge), the name of the earring (e.g., Jane Doe's earring), and/or other data to a computing system (e.g., a smartphone) using a wireless communication and/or protocol. The smartphone can then perform further data analysis, such as: compare the external ear temperature to the ambient temperate; and determine physiological, psychological, and/or emotional events.

Unfortunately, wireless communication circuitry and/or microcontrollers may be more power-intensive than the temperature sensors (e.g., first temperature sensor 510, second temperature sensor 512). Therefore, the wireless communication circuitry and/or the microcontroller can shorten the battery life of a wearable device. Note that wireless communication includes transmitting high-frequency radio frequency (RF) signals, for example, in the GHz range of RFs.

Some wearable devices or some Internet of Things (IoT) devices may utilize backscatter communication to sending data to a computing system. This communication method, however, may require a customized carrier wave transmitter or a customized radio receiver, which may increase the difficulty of the wearable device or the IoT device to communicate directly to a user's computing system (e.g., a user's smartphone). To simplify the wireless communication of the temperature data between the earring 500 of a user and the user's computing system (e.g., the user's smartphone), in some embodiments, the earring 500 may utilize a Bluetooth microcontroller. Therefore, in some embodiments, the wireless communication circuitry 514 may be a Bluetooth (e.g., Bluetooth Classic®; a Bluetooth Low Energy® or BLE®) microcontroller.

In some embodiments, the earring 500 may be built using an NRF52832 microcontroller, where the NRF52832 microcontroller serves as the wireless communication circuitry 514 of the earring 500. Specifically, the NRF52832 microcontroller is a system-on-chip (SoC) manufactured by Nordic Semiconductor. The NRF52832 may be based on a 32-bit ARM Cortex-M4 processor with a floating point for computing tasks. The NRF52832 may be designed for a Bluetooth Low Energy® (or BLE®) communication application(s) and/or other 2.4 GHz wireless communication applications.

In some embodiments, the earring 500 may utilize the wireless communication circuitry 514 (e.g., the NRF52832, Bluetooth Classic® microcontroller, BLE® microcontroller, etc.) in an advertising mode. The advertising mode may be a feature that configures the earring 500 utilizing the BLE® microcontroller to broadcast the presence and/or identity of the earring 500 to other devices that utilize a Bluetooth microcontroller, such as a smartphone (e.g., the computing system 116 of FIG. 1). Using the advertising mode of the BLE® microcontroller, the earring 500 can embed customized data (e.g., temperature sensor data, earring name, wearable device identifier, battery charge level) of, for example, 31 bytes in short advertising packets. Bluetooth advertising consumes significantly lower power compared to establishing a continuous Bluetooth connection. A continuous Bluetooth connection requires sending a series of packets to synchronize and/or negotiate the frequency hopping sequence, as well as regular wireless transmissions of the series of packets to keep the wireless communication connection “alive” or active. Therefore, the advertising mode used by the wireless communication circuitry 514 (e.g., the NRF52832, Bluetooth Classic® microcontroller, BLE® microcontroller, etc.) of the earring 500 may increase the battery 508 life of the earring 500.

In some embodiments, the voltage may decrease (e.g., a voltage drop) when a wireless communication circuitry transmits a packet due to the current limitations of, for example, the battery 508 and/or the wireless communication circuitry 514 of the earring 500. Therefore, implementing a power cycling and/or operating the wireless communication circuitry 514 in the advertising mode and/or with sufficient time between data packets (e.g., temperature sensor data, earring name, wearable device identifier, battery charge level) allows the voltage of the battery 508 and/or the wireless communication circuitry 514 to recover prior to the transmission of the next data packet.

In some embodiments, each data point sent by the earring 500 can be compressed into two bytes. This compression can be more energy-efficient to transmit the data using the Bluetooth advertising mode, instead of maintaining a constant Bluetooth wireless communication connection with the computing system 116 of FIG. 1 (e.g., a user's smartphone).

In some embodiments, the wireless communication circuitry 514 (e.g., the NRF52832, Bluetooth Classic® microcontroller, BLE® microcontroller, etc.) may communicate with the two temperature sensors (e.g., first temperature sensor 510, second temperature sensor 512) using I2C, and the wireless communication circuitry 514 can pack the temperature data into advertising packets.

In some embodiments, these data packets may adhere to a Bluetooth advertising structure, such as: beginning with a fixed preamble pattern, an access address, a header, a payload, a cyclic redundancy check (CRC), or combinations thereof. In some embodiments, the payload of the Bluetooth packet may contain the temperature data, an energy level information of the power source 508, and/or a customized name or other identifier of the earring 500 (e.g., Jane Doe's earring). In some embodiments, the data may be wrapped as service data, starting with a two-byte universally unique identifier (UUID) of 0x1809, which may correspond to health temperature data in the Bluetooth protocol. The customized name of the earring 500 typically may range from four to ten bytes.

In some embodiments, the wireless communication circuitry 514 of the earring 500 can be configured to transmit advertising packets at different intervals to balance the need for visibility with power consumption. Therefore, a manufacturer of the earring 500 can conduct experiments to explore how different advertising intervals of the wireless communication circuitry 514 can affect the battery 508 life of the earring 500. For example, by changing the intervals of the transmission of the advertising packets, the manufacturer can increase or decrease the battery 508 life of the earring 500. That is, the shorter the time interval between a first and a second transmission of the advertising packets, the shorter the battery 508 life. And, vice versa, the longer the time interval between the first and the second transmission of the advertising packets, the longer the battery 508 life. Therefore, there is a trade-off between a transmission frequency and the battery 508 life. For example, sensing the temperatures, using the earring 500, every ten seconds can significantly increase the battery 508 life of the earring 500 compared to sensing the temperatures every one (1) second.

In the development of wearable devices, such as the earring 500, the power source may need to supply a high-power capacity, while being compact and/or lightweight. In some embodiments, the example earring 500 may be built using a Seiko MS621FE lithium manganese battery (e.g., battery 508), which is a rechargeable battery. Note that the example earring 500 is not a limiting example. The MS621FE rechargeable battery may offer a capacity of approximately 5.5 mAh; has a diameter of approximately 6.8 mm diameter; weighs approximately 0.23 grams; can generate a maximum output voltage of 3V; has a discharge current of approximately 15 uA; and has a maximum continuous discharge current of approximately 0.25 mA.

In some embodiments, the MS621FE may be capable to support and/or power the first temperature sensor 510, the second temperature sensor 512, and the wireless communication circuitry 514 (e.g., (e.g., the NRF52832, Bluetooth Classic® microcontroller, BLE® microcontroller, etc.) in a “sleep” mode. Unfortunately, in some embodiments, the MS621FE may not be sufficient to robustly startup and/or transmit Bluetooth packets, which both may require 5 mA of current. Fortunately, both, the startup and/or the Bluetooth transmissions are transient operations that only require short pulses of high current. Therefore, in some embodiments, the earring 500 may include a capacitor of, for example, 100 uF, where the capacitor is coupled in parallel with the MS621FE (e.g., the battery 508). The capacitor buffers charge, while the earring 500 and/or the wireless communication circuitry 514 is in “sleep” mode. The capacitor can then provide a pulse of mA-level current by quickly discharging itself when needed.

From the foregoing it will be appreciated that, although specific embodiments have been described herein for purposes of illustration, various modifications may be made while remaining within the scope of the claimed technology. Examples described herein may refer to various components as “coupled” or signals as being “provided to” or “received from” certain components or nodes. It is to be understood that in some examples the components are directly coupled one to another, while in other examples, the components are coupled with intervening components disposed between them.

Similarly, signals or communications may be provided directly to and/or received directly from the recited components without intervening components, but also may be provided to and/or received from the certain components through intervening components.

What is claimed is:

1. A wearable device comprising:
a first temperature sensor configured to be positioned to detect an external ear temperature at an ear;
a second temperature sensor configured to dangle below the ear to detect an ambient temperature;
a controller coupled to the first temperature sensor and the second temperature sensor; and
an antenna coupled to the controller, wherein the controller and the antenna are configured to communicate data indicative of the external ear temperature and the ambient temperature.

2. The wearable device of claim 1, wherein the first temperature sensor is positioned on, or embedded in, an auricle of the ear, and wherein the external ear temperature is correlated to a corporeal temperature of a user of the wearable device.

3. The wearable device of claim 2, wherein the first temperature sensor is positioned on, or embedded in, an earlobe of the ear.

4. The wearable device of claim 1, wherein the first temperature sensor is positioned outside an ear canal of the ear.

5. The wearable device of claim 1, wherein the first temperature sensor, the second temperature sensor, the controller, and the antenna are packaged into an earring wearable on the ear.

6. The wearable device of claim 5, wherein:
the earring further comprises a power source; and
the first temperature sensor, the second temperature sensor, and the controller are configured to be powered by the power source.

7. The wearable device of claim 1, wherein the controller is configured to implement power cycling.

8. The wearable device of claim 6, wherein the controller and the antenna are configured to communicate the data using Bluetooth advertising packets.

9. The wearable device of claim 8, wherein the Bluetooth advertising packets comprise:
a time interval between a first data packet of the data and a second data packet of the data; and
the time interval causes a voltage of the power source to recover prior to a transmission of the second data packet.

10. The wearable device of claim 9, wherein at least one of the first or second data packets further comprises an energy level information of the power source, a name of the earring, or combinations thereof.

11. The wearable device of claim 1, wherein the antenna and the controller are configured to communicate the data to a computing system, and wherein the computing system is configured to detect an event based in part on the external ear temperature and the ambient temperature.

12. The wearable device of claim 5, wherein the earring is manufactured using a flexible printed circuit board (FPCB), and wherein:
the first temperature sensor is embedded in or on a first portion of the earring;
the second temperature sensor is embedded in or on a second dangling portion of the earring; and
the FPCB allows the second dangling portion of the earring to move as a user of the earring moves their head or body.

13. The wearable device of claim 11, wherein the event comprises eating, exercising, stress, excitement, meditating, praying, sleeping, ovulation, vasomotor symptoms (VMS), hypothyroidism, sepsis, hypoglycemia, malnutrition, adrenal insufficiency, extensive burns, an infection, hyperthyroidism, an autoimmune disease, malignant hyperthermia, an endocrine disorder, or combinations thereof.

14. A computing system comprising:
a processor;
at least one computer readable media coupled to the processor, the computer readable media encoded with instructions which, when executed by the processor, cause the computing system to perform operations comprising:
receive data indicative of an external ear temperature from a wearable device positioned on an ear of a user;
receive data indicative of an ambient temperature from the wearable device; and
detect an event based on the data indicative of the external ear temperature and the data indicative of the ambient temperature.

15. The computing system of claim 14 further comprising:
a display; and
the computing system performs further operations comprising:
display, on the display, the event, instructions to a user, the external ear temperature, the ambient temperature, a first graph of the external ear temperature over time, a second graph of the ambient temperature over time, or combinations thereof.

16. The wearable device of claim 14, wherein the event comprises eating, exercising, stress, excitement, meditating, praying, sleeping, ovulating, vasomotor symptoms (VMS), hypothyroidism, sepsis, hypoglycemia, malnutrition, adrenal insufficiency, extensive burns, an infection, hyperthyroidism, an autoimmune disease, malignant hyperthermia, an endocrine disorder, or combinations thereof.

17. The computing system of claim 14, wherein the instructions comprise a machine learning model or heuristics for the detection of the event.

18. A method comprising:
wearing one or more earrings, each earring having:
a first portion abutting an external ear, the first portion including a temperature sensor positioned to detect a temperature of the external ear; and
a second portion dangling below the external ear, the second portion including a second temperature sensor positioned to detect an ambient temperature;
transmitting data indicative of the temperature of the external ear and the ambient temperature to a computer system;
reviewing an event displayed by the computer system, the event detected based on the data.

19. The method of claim 18, wherein the transmission comprises a wireless transmission.

20. The method of claim 18, wherein the event comprises a physiological state, a psychological state, an emotional state, or combinations thereof of a user of the one or more earrings.
